(19) United States
(12) Patent Application Publication
(10) Pub. No.: US 2026/0115488 A1
(43) Pub. Date: Apr. 30, 2026

(54) NEEDLE CATHETER

(71) Applicant: Rakuten Medical, Inc., San Diego, CA (US)

(72) Inventors: Masami KAWASHIMA, Tokyo (JP); Roland BAYS, Ecublens (CH)

(21) Appl. No.: 19/433,105

(22) Filed: Dec. 26, 2025

Related U.S. Application Data
(63) Continuation of application No. PCT/US2024/035922, filed on Jun. 27, 2024.

(30) Foreign Application Priority Data
Jun. 30, 2023 (JP) 2023-107888

(51) Int. Cl. A61N 5/06; A61L 2/20; A61L 29/06; A61L 103/15; A61N 5/067

(52) U.S. Cl. CPC A61N 5/0601; A61L 2/20; A61L 29/06; A61N 5/062; A61N 5/067; A61L 2103/15; A61L 2202/15; A61N 2005/0612; A61N 2005/063

(57) ABSTRACT

The catheter includes a needle 2 that is punctured into tissue and an obturator 3 that is extractably inserted with respect to the needle, the needle has a hollow needle body 2a with a pointed distal end portion 2t and a needle hub 2b that supports a proximal end portion 2e, the interior of the needle body includes a single lumen 2p continuous from the proximal end portion to the distal end portion and a closed end 2c that closes the lumen, and the closed end has a contoured shape that is rounded and continuous in the shape of an arc.

### CROSS-REFERENCE TO RELATED APPLICATIONS

This application is a Continuation Application of PCT Application No. PCT/US2024/035922, filed Jun. 27, 2024 and based upon and claiming the benefit of priority from prior Japanese Patent Application No. 2023-107888, filed Jun. 30, 2023, the entire contents of which are incorporated herein by reference.

### BACKGROUND OF THE INVENTION

### 1. Field of the Invention

The present invention relates to a needle catheter for medical use.

### 2. Description of the Related Art

Currently, this type of needle catheter comprises, for example, a resin needle and a stainless steel obturator, and is used for introducing into tissue a diffuser for interstitial irradiation, which is configured by optical fibers.

When using the catheter, first, the needle is punctured into the tissue while the obturator is kept inserted into the lumen of the needle. After that, the obturator is removed and only the needle is retained in the tissue. The diffuser for interstitial irradiation is then inserted into the lumen of the needle that is retained in the tissue. In this state, a laser beam emitted from the diffuser is irradiated through the needle into the tissue, thereby performing light therapy in the tissue.

Needle catheters are used in the medical field for the purpose of maintaining, restoring, and improving human health, for example, in the environment of diagnosis and treatment of diseases. Therefore, needle catheters are required to maintain a certain level of quality in terms of durability, sterilizability, identifiability, non-degradability, and the like.

As a quality related to durability, the lumen contour of a conventional needle tip has an angular contour shape (e.g., a rectangular inner surface shape) with a steeply varying angle. According to such a contour shape, for example, when an external force acts on the needle during diagnosis or treatment, stress is easily concentrated in the angular portion of the tip lumen. Depending on the strength of the stress, the tip of the needle may rupture or break prematurely. Therefore, a certain level of durability is required to prevent this from happening.

As a quality related to sterilizability, needle catheters are configured by two components (needle and obturator) joined in a luer taper relationship. According to such a joint condition, it is difficult to ensure that the sterilizing gas is distributed evenly over the entire front and back surfaces of each component. In that case, there is a risk that insufficiently sterilized portions may remain in, for example, a joining area where no gaps are formed due to the luer taper relationship, or a needle lumen area where the obturator is inserted. Therefore, a certain level of sterilizability is required to prevent this from happening.

As a quality related to identifiability, needle catheters are prepared in advance in multiple types with different lengths. A needle catheter with an optimum length is selected according to the amount of puncture (depth of puncture) into the tissue in diagnosis and treatment. In this case, it is difficult to accurately identify the needle catheter with the optimum length from among the multiple types in a short period of time. In the case where the catheter is incorrectly selected, the diagnosis or treatment may have to be redone. Therefore, a certain level of identifiability is required to prevent this from happening.

As a quality related to non-degradability, among the needle catheter components (needle and obturator), the needle is configured by two parts (needle hub and long needle body) made of polyacetal (POM), which is an engineering plastic, that are coupled to each other. Polyacetal (POM) is a material (synthetic resin) that is extremely difficult to bond to other things by itself. In this case, depending on the coupled state, the parts may come apart from each other, and, for example, the needle body may fall off the needle hub. Therefore, a certain level of non-degradability is required to prevent this from happening.

### BRIEF SUMMARY OF THE INVENTION

One of the purposes of the present invention is to meet the requirements mentioned above and provide a needle catheter capable of maintaining a certain level of quality in terms of durability, sterilizability, identifiability, non-degradability, and the like.

To achieve such a purpose, a needle catheter according to embodiments comprises a needle that is to be punctured into tissue and an obturator that is extractably inserted with respect to the needle, in which the needle includes a hollow and long needle body that is molded of a translucent resin material and provided with a pointed and closed distal end portion and a needle hub that supports a proximal end portion of the needle body on an opposite side of the distal end portion, the interior of the needle body is configured to include one lumen that continuously extends from the proximal end portion to the distal end portion and a closed end that closes the lumen at the distal end portion so as to become an end of the lumen, and the closed end has a rounded, arcuate, continuous contour shape.

According to the above structure, it is possible to realize a needle catheter capable of maintaining a certain level of quality in terms of durability, sterilizability, identifiability, non-degradability, and the like. The embodiments of the needle catheter described herein are useful in combination with a diffuser for emitting light. The embodiments may be used for treatment of a tumor or lesion, such as a cancerous or precancerous lesion. The embodiments may be used in combination with a light-activatable therapy or light-activatable drug for treatment, such as for treatment of a cancer, tumor or lesion, such as a cancerous or precancerous lesion.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is an overall perspective view of a needle catheter according to one embodiment of the present invention.

FIG. 2 is an overall perspective view of an obturator which is a component of the needle catheter of FIG. 1.

FIG. 3 is an overall perspective view of a needle which is a component of the needle catheter of FIG. 1.

FIG. 4 is a cross-sectional view of an obturator and a needle joined in a luer taper relationship.

FIG. 5 is a cross-sectional view of a distal end portion of the needle.

FIG. 6 is a perspective view of a locking collar with an identification mark.

FIG. 7 is a cross-sectional view of a groove structure configured through a joining area between the obturator and the needle, joined in the luer taper relationship.

### DETAILED DESCRIPTION OF THE INVENTION

### Description of Embodiment

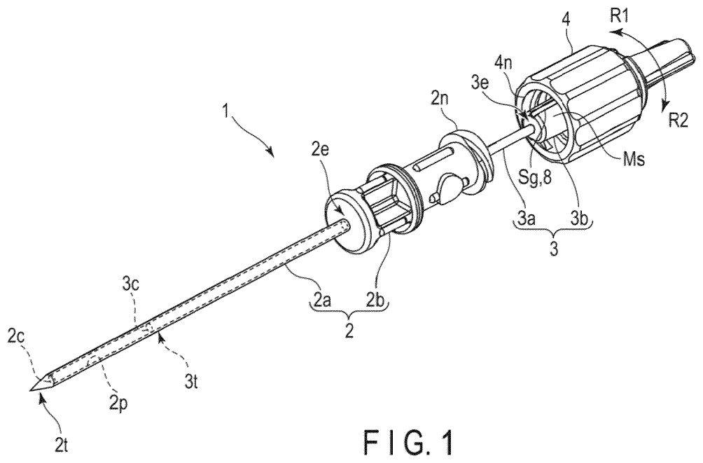

FIG. 1 is an overall configuration view of a needle catheter 1. The needle catheter 1 is used to introduce a diffuser for interstitial irradiation (not shown), which is configured by optical fibers, into tissue during interstitial treatment. Note that the needle catheter 1 of the present embodiment can be applied to any type of diffuser, for example, not only a surface irradiation diffuser that irradiates from the front of the optical fibers, but also a side irradiation diffuser that irradiates from the side surface of the optical fibers.

As shown in FIG. 1, the needle catheter 1 comprises a resin needle 2 that is punctured into the tissue, a stainless steel obturator 3 that is extractably inserted with respect to the needle 2, and a locking collar 4 that mutually secures the obturator 3 and the needle 2 when the obturator 3 is inserted into the needle 2.

The locking collar 4 is molded from polypropylene resin and is rotatably mounted on the obturator 3 in the direction of arrows R1 and R2. The locking collar 4 is provided with a threaded portion 4n on its inner circumference. On the other hand, the needle 2 is provided with a threaded portion 2n on its outer circumference (specifically, on the outer circumference of a needle hub 2b, which will be described later).

In this case, in a state where the obturator 3 is inserted into the needle 2, the locking collar 4 is rotated in the direction of arrow R1. At this time, both threaded portions 2n and 4n are screwed together, and the locking collar 4 is fastened to the needle 2. As a result, the obturator 3 and the needle 2 are mutually secured.

After this, the locking collar 4 is rotated in the direction of arrow R2 (i.e., in the opposite direction of arrow R1). At this time, the locking collar 4 is released from the needle 2 by the threads 2n and 4n on both sides being unscrewed. As a result, the obturator 3 can be withdrawn from the needle 2 together with the locking collar 4.

Here, as a preparation before using the needle catheter 1, the obturator 3 is inserted into the needle 2, and the locking collar 4 is fastened to the needle 2 as described above. This mutually secures (i.e., integrates) the obturator 3 and the needle 2. At this time, the needle 2 is given the rigidity of the obturator 3 itself. This allows the needle 2 to maintain a contour shape that matches the preset contour shape of the obturator 3 itself.

The needle 2 is then punctured into the tissue. After this, the obturator 3 is withdrawn together with the locking collar 4, and only the needle 2 is retained in the tissue. Then, the diffuser for interstitial irradiation is inserted into the needle 2 that has been retained in the tissue.

The needle body is constructed with translucent tubing, in some cases, white translucent tubing. The translucent feature increases the light uniformity at the surface of the needle such as by reflecting the light emitted from the diffuser multiple times by the walls of the needle before the light is then emitted from the needle. The translucent property may be further modified to control for light scattering and to achieve the desired illumination. Such material includes polyacetal (POM). In this state, when light is emitted from the diffuser, the emitted laser light penetrates the needle 2 and the light is irradiated into the tissue from the needle 2 that has been retained in the tissue. As a result, light therapy is performed in the tissue in the specific vicinity of the inserted needle.

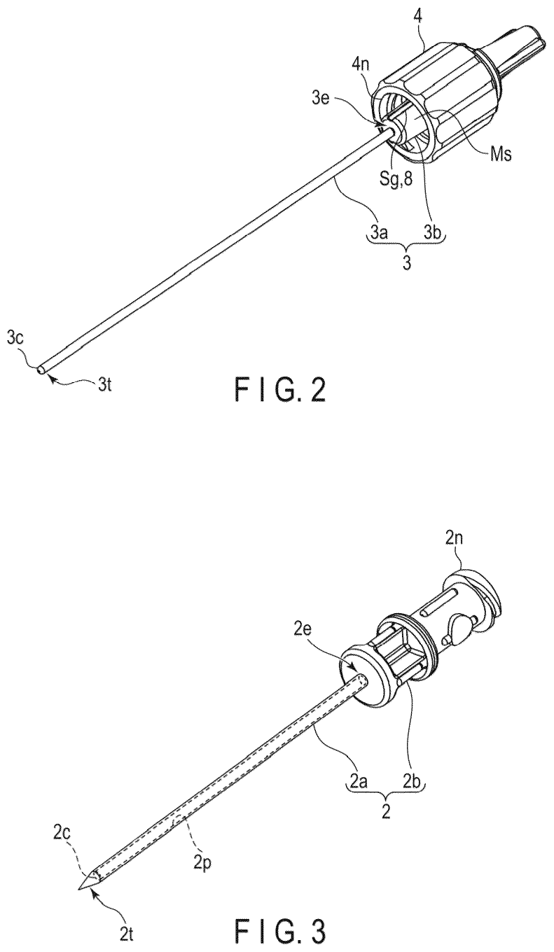

FIG. 2 is an overall configuration view of the stainless steel obturator 3. The obturator 3 includes an obturator body 3a and an obturator hub 3b. The obturator body 3a has a preset contour shape. The obturator hub 3b has a generally cylindrical shape with a larger diameter than the obturator body 3a.

As shown in FIG. 2, the obturator body 3a is rigid enough to maintain the preset contour shape. As an example of the preset contour shape in FIG. 2, the obturator body 3a has a solid and long, elongated cylindrical shape extending from a proximal end portion 3e to a distal end portion 3t. Note that the shape of the obturator main body 3a includes, for example, a straight extended shape, a bent and extended shape, and an arc-like extended shape.

The solid obturator body 3a is circular in a cross-sectional view, and its diameter (outer diameter) is set at a constant value from the proximal end portion 3e to the distal end portion 3t. The obturator body 3a comprises a rounded arc-shaped contact end 3c at the distal end portion 3t.

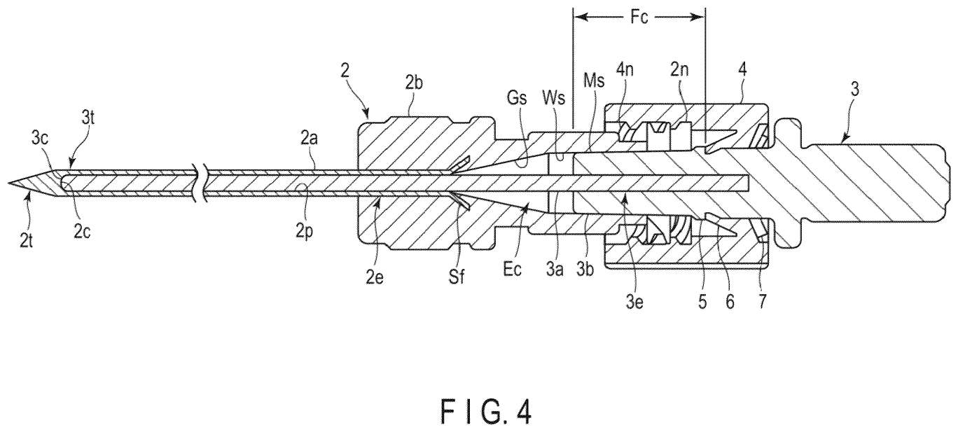

The obturator body 3a is extractably inserted from the distal end portion 3t into the needle 2 (specifically, into a lumen 2p of a hollow needle body 2a (see FIG. 3 and FIG. 4), which will be described later). In a state where the obturator body 3a is inserted into the needle 2, the contact end 3c of the distal end portion 3t is configured to be able to come into surface contact with a closed end 2c (see FIG. 3 and FIG. 4) of the needle body 2a, which will be described later.

As shown in FIG. 2, the proximal end portion 3e of the obturator body 3a is provided on the opposite side of the distal end portion 3t and is supported by (coupled to) the obturator hub 3b. Note that, as a supporting (coupling) method, for example, the obturator body 3a and the obturator hub 3b may be integrally molded in a series of manufacturing processes, or the obturator body 3a and the obturator hub 3b may be manufactured separately and then coupled to each other, e.g., mechanically joined or glued together, in a post-installation process.

A male tapered surface Ms is formed along the outer circumference of the portion of the obturator hub 3b that supports (couples) the proximal end portion 3e of the obturator body 3a. The male tapered surface Ms has a conical shape with a tapering gradient of, for example, approximately 6% toward the distal end portion 3t of the obturator body 3a.

The male tapered surface Ms is configured so that when the obturator 3 is inserted into the needle 2, it can be joined (contacted) with a female tapered surface Ws of the needle 2 (see FIG. 4), which is described below, without any gap. At this time, a joining area Fc (see FIG. 4) is formed in which the male tapered surface Ms and the female tapered surface Ws are joined without any gap, whereby the obturator 3 and the needle 2 are firmly joined in a luer taper relationship.

Note that luer taper is a system applied to standardized small-scale fluid connectors, and, for example, is used for establishing a leak-free connection between a male taper connection site and a paired female taper connection site.

Furthermore, the above-described locking collar 4 is mounted on the obturator 3 in a manner not to fall off by means of a fall-off prevention structure (see FIG. 4) described later. As a result, as shown in FIG. 2, the locking collar 4 can be rotated in the direction of arrows R1 and R2 without falling off from the obturator 3.

FIG. 3 is an overall configuration view of the resin needle 2. The needle 2 includes the needle body 2a and the needle hub 2b. The needle body 2a is molded from a translucent resin material, and its entirety is configured elastically deformable. The needle hub 2b has a generally cylindrical shape with a larger diameter than the needle body 2a.

As shown in FIG. 3, the needle body 2a has a hollow, long, elongated cylindrical shape extending from a proximal end portion 2e to a distal end portion 2t. The hollow needle body 2a has a single lumen 2p configured therein that is continuously extending from the proximal end portion 2e to the distal end portion 2t. The lumen 2p is circular in a cross-sectional view, and its diameter (inside diameter) is set to a constant value from the proximal end portion 2e to the distal end portion 2t.

As shown in FIG. 2 and FIG. 3, the diameter (inner diameter) of the lumen 2p of the needle body 2a can be set based on the diameter (outer diameter) of the obturator body 3a described above. In this case, it is preferable that the diameter (inner diameter) of the lumen 2p of the needle body 2a is set in a manner that the obturator body 3a can be moved smoothly along the lumen 2p of the needle body 2a when the obturator 3a is inserted or withdrawn from the needle 2.

Note that, as an example of the setting method, the following three variations are assumed. As a first variation, the diameter (inner diameter) of the lumen 2p of the needle body 2a is set slightly larger than the diameter (outer diameter) of the obturator body 3a described above. As a second variation, the diameter (inner diameter) of the lumen 2p of the needle body 2a is set slightly smaller than the diameter (outer diameter) of the obturator body 3a. As a third variation, the diameter (inner diameter) of the lumen 2p of the needle body 2a is set to match the diameter (outer diameter) of the obturator body 3a described above.

The distal end portion 2t of the needle body 2a having such a lumen 2p is pointed and closed. Inside this closed distal end portion 2t, the closed end 2c which closes the lumen 2p to become the end of the lumen 2p is configured.

Here, the contour shape of the closed end 2c is preferably set to be rounded and continuous in the shape of an arc, similar to the contour shape of the contact end 3c provided at the distal end portion 3t of the obturator body 3a described above. For example, in a case where the mutual contour shapes of the closed end 2c and the contact end 3c are set to be arc-shaped, the curvature (or radius of curvature) of both ends should be set to values that are consistent with each other. In some cases, the radius (R) of the arc is no less than 0.35 mm.

Note that the needle body 2a is configured such that the above-described proximal end portion 2e provided on the opposite side of the distal end portion 2t is supported by the needle hub 2b. The needle hub 2b is provided with the threaded portion 2n on its outer circumference, which can be screwed with the threaded portion 4n provided on the inner circumference of the locking collar 4 described above.

In this case, in a state where the obturator 3 (obturator body 3a) is inserted into the needle 2 (lumen 2p of the needle body 2a) and the two are joined (see FIG. 4), the contact end 3c of the obturator body 3a and the closed end 2c of the needle body 2a are in surface contact.

FIG. 4 shows a joint condition of the obturator 3 and the needle 2. The needle hub 2b is provided with an annular joining space Ec continuous with the lumen 2p of the needle body 2a. In the joining space Ec, when the obturator 3 is inserted into the needle 2, the obturator body 3a is inserted toward the lumen 2p of the needle body 2a, and the obturator hub 3b and the needle hub 2b are mutually joined.

As shown in FIG. 4, inside the needle hub 2b is provided with the female tapered surface Ws configured along its inner circumference and a guide surface Gs continuous from the female tapered surface Ws to the lumen 2p of the needle body 2a. The joining space Ec is configured over the space area surrounded by these female tapered surface Ws and guide surface Gs.

The female tapered surface Ws has a conical shape with a tapering slope of, for example, approximately 6% toward the distal end portion 2t of the needle body 2a. In this case, the female tapered surface Ws on the inner circumference of the needle hub 2b and the male tapered surface Ms on the outer circumference of the above-mentioned obturator hub 3b form a conical shape having the same tapered slope as each other.

The guide surface Gs forms a conical shape having a tapering slope from the female tapered surface Ws toward the lumen 2p of the needle body 2a. The guide surface Gs guides the distal end portion 3t of the obturator body 3a toward the lumen 2p of the needle body 2a when the obturator 3 is inserted into the needle 2. At this time, the distal end portion 3t of the obturator body 3a moves along the guide surface Gs while contacting the guide surface Gs. This allows the distal end portion 3t of the obturator body 3a to be smoothly and securely inserted into the lumen 2p of the needle body 2a, so that the obturator body 3a is smoothly inserted into the lumen 2p of the needle body 2a.

On the other hand, the threaded portion 2n is provided on the outside of the needle hub 2b along its circumferential direction, and the threaded portion 2n is configured so that the threaded portion 4n of the locking collar 4 described above can be screwed together. The locking collar 4 is mounted on the obturator 3 by a fall-off prevention structure described below. The locking collar 4 is mounted to be rotatable in the direction of arrows R1 and R2 along the outer circumference of the obturator hub 3b.

In FIG. 4, as an example of the fall-off prevention structure, on the outside of the obturator hub 3b is provided an annular stopper piece 5 continuously protruding along its outer circumference direction. In another example, the inside the locking collar 4 is provided an annular engaging piece 6 continuously protruding along its inner circumference direction.

In another example of the fall-off prevention, a force spring 7 is provided outside the obturator hub 3b, and by this force spring 7, the engaging piece 6 of the locking collar 4 is always maintained in a state of being pressed toward the stopper piece 5 of the obturator hub 3b. In this configuration, the engaging piece 6 of the locking collar 4 is in a state of being rotatably held between the stopper piece 5 and the force spring 7. Note that the force (i.e., the pressing force) of the force spring 7 is set to a level that does not degrade the rotational performance of the locking collar 4. The spring force of force spring 7 is exerted by tightening the obturator 3, which also allows obturator 3 to push the needle 2 against the closed end 2c. This could be also achieved by mechanical latches.

As a result, the locking collar 4 can be smoothly rotated in the direction of arrows R1 and R2 (see FIG. 2) while remaining in a state of being mounted on the obturator hub 3b without falling off (i.e., without coming off from the obturator 3).

In this case, as shown in FIG. 4, the locking collar 4 is fastened to the needle hub 2b by rotating the locking collar 4 in the direction of arrow R1 in a state where the obturator 3 (obturator body 3a) is inserted into the needle 2 (lumen 2p of the needle body 2a). The needle hub 2b is pulled into the obturator hub 3b in response to the rotation of the locking collar 4. This maintains and configures the joining area Fc where the male tapered surface Ms and the female tapered surface Ws are joined without any gap. The joining area Fc has a tapered conical shape toward the direction in which the obturator 3 is inserted into the needle 2.

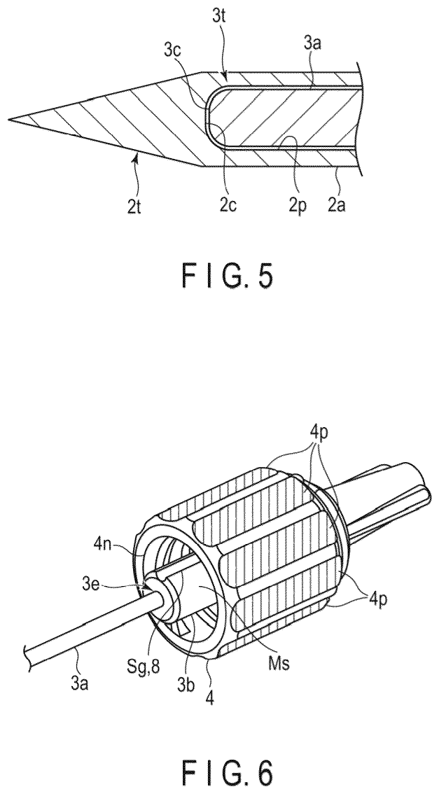

As shown in FIG. 4 and FIG. 5, the contact end 3c of the obturator body 3a makes a surface contact with the closed end 2c of the needle body 2a in response to the rotation of the locking collar 4. An external force from the contact end 3c of the obturator body 3a acts in a uniformly distributed manner along the closed end 2c of the needle body 2a. Thus, the rigidity of the obturator body 3a is uniformly imparted to the needle body 2a. As a result, the needle body 2a is maintained in a straight, elongated cylindrical shape while being elastically deformed into a shape that follows the contour of the obturator body 3a.

FIG. 6 is an external view of the locking collar 4 with an identification mark. The identification mark identifies the type of needle catheter 1 (i.e., needle 2). For example, coloring, uneven marks, symbols, numbers, letters, etc. are assumed as identification marks, all of which can be specified as visually identifiable by humans. For example, the type of needle 2 to be identified by such an identification mark is assumed to be the total length of the needle body 2a in a state where the obturator body 3a is inserted.

As shown in FIG. 6, the identification mark can be added to a part of or all of the outer surface of the locking collar 4. In FIG. 6, as an example, an identification mark (hatched indication) is added to a plurality of arcuate recesses 4p arranged at equal intervals along the outer surface of the locking collar 4. As the identification mark, different coloring is added according to differences in the overall length of the needle body 2a.

For example, assuming two types of needle bodies 2a (needles 2) having different overall lengths, different coloring is added to the locking collar 4 mounted on the obturator 3 to be inserted into each needle 2. For identifications marks, an arcuate recess 4p of one locking collar 4 is colored red, and an arcuate recess 4p of the other locking collar 4 is colored blue.

This makes it possible to easily, quickly, and accurately identify the type of needle 2, i.e., the total length of the needle body 2a, just by looking at the locking collar 4. Note that, as an identification mark, the above coloring, uneven marks, symbols, numbers, letters, etc. may be added in combination with each other.

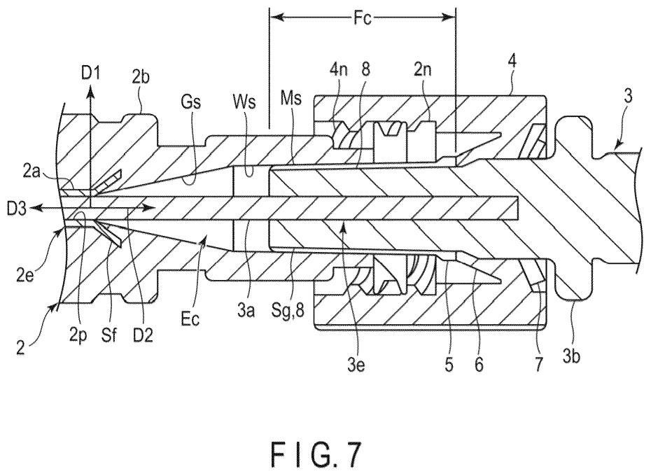

FIG. 7 is a cross-sectional configuration view of a groove structure Sg for distributing sterilizing gas.

The needle catheter 1 of the present embodiment is sterilized in its entirety by exposing it to an atmosphere of sterilizing gas. In this case, it is necessary for the sterilizing gas to spread to every corner of the needle catheter 1 without leakage. For example, it is necessary to ensure that the sterilizing gas is evenly distributed from between the obturator 3 and the needle 2, which are joined to each other in a luer taper relationship (i.e., the joining area Fc), to also the gap between the obturator body 3a and the needle body 2a.

However, since the male tapered surface Ms and the female tapered surface Ws are joined without any gap in the joining area Fc, there is a risk that the sterilizing gas will not be able to spread between these surfaces Ms and Ws.

Therefore, the groove structure Sg is provided to distribute the sterilizing gas. The groove structure Sg is configured by penetrating a part of the joining area Fc described above. In the example of FIG. 7, the groove structure Sg is provided in the obturator hub 3b and is configured by arranging a plurality of grooves 8 in a manner penetrating the joining area Fc.

As shown in FIG. 7, the plurality of grooves 8 are configured by partially depressing the outer circumference of the obturator hub 3b, which is the joining area Fc. The plurality of grooves 8 are each arranged along the insertion direction of the obturator 3 with respect to the needle 2 (parallel, substantially parallel) and spaced apart along the circumferential direction.

In this case, the plurality of grooves 8 may be arranged at equal intervals along the circumferential direction, or may be arranged at unequal intervals (randomly). Furthermore, it is preferable that the depth and size of the grooves 8 is set to a degree that does not affect the joining accuracy of the needle 2 (specifically, the needle hub 2b) and the obturator 3 (specifically, the obturator hub 3b) in the joining area Fc. In one embodiment, groove is made with Φ=0.8±0.7 mm, such that the depth of the groove is 0.4±0.35 mm and the width of the groove is 0.8±0.7 mm. Furthermore, the shape of the grooves 8 can be set arbitrarily, for example, arc-shaped in cross-section, triangular-shaped in cross-section, and rectangular-shaped in cross-section.

According to such a groove structure Sg, in a state where the needle catheter 1 is exposed to a sterilizing gas atmosphere, the sterilizing gas flows through the joining area Fc where the groove structure Sg is configured, and the entire front and back surfaces of both needle 2 and obturator 3 are sterilized.

FIG. 7 shows a flair structure Sf that couples the proximal end portion 2e of the needle body 2a to the needle hub 2b. The flair structure Sf is applied to the proximal end portion 2e of the needle body 2a inside the needle hub 2b. As an example in FIG. 7, the flair structure Sf is configured to protrude outward from the needle body 2a at the proximal end portion 2e of the needle body 2a. In another embodiment, the needle hub 2b is configured by a resin material molded to cover the flair structure Sf.

Here, the protruding form of the flair structure Sf is defined by a first direction D1 orthogonal to the needle body 2a, a second direction D2 from the distal end portion 2t to the proximal end portion 2e of the needle body 2a, and a third direction D3 from the proximal end portion 2e to the distal end portion 2t of the needle body 2a.

The flair structure Sf shown in FIG. 7 is in the form of a flair structure protruding outward from the proximal end portion 2e of the needle body 2a toward a direction between the first direction D1 and the second direction D2. In this case, the protruding form of the flair structure Sf protrudes continuously along the outer circumference of the needle body 2a, or may protrude intermittently along the outer circumference of the needle body 2a.

In either form, the needle hub 2b is insert molded to cover the flair structure Sf of the proximal end portion 2e of the needle body 2a. For example, in a state where the proximal end portion 2e of the needle body 2a, which has a flair structure Sf in advance, is set in a mold (not shown), resin for needle hub molding is injected to cover its surroundings, and they are integrally molded together (composite molding). In such configuration, the flair structure Sf bites into the resin-molded needle hub 2b, thereby coupling the needle body 2a to the needle hub 2b.

Features and advantages of the described needle catheter embodiments

In the embodiments described herein, inside the distal end portion 2t of the needle body 2a, the contour shape of the closed end 2c, which is the end of the lumen 2p, is set to be rounded and continuous in an arc shape. In this case, it is possible to eliminate a portion where the angle changes sharply as in the conventional type of needle catheter. The embodiments described herein allows stress to be dispersed along the arc-shaped closed end 2c. As a result, it is possible to prevent problems such as rupture, breakage, and damage of the distal end portion 2t of the needle body 2a.

In the embodiments described herein, the distal end portion 3t of the obturator body 3a is provided with a rounded, arc-shaped contact end 3c, and the distal end portion 2t of the needle body 2a is provided with a rounded, continuously arc-shaped closed end 2c, which is similar to the contour shape of the contact end 3c. Therefore, in a state where the obturator 3 (obturator body 3a) is inserted into the needle 2 (lumen 2p of the needle body 2a) and the two are joined, the contact end 3c of the obturator body 3a and the closed end 2c of the needle body 2a are in surface contact. In this case, the external force from the contact end 3c of the obturator body 3a acts in a uniformly distributed manner along the closed end 2c of the needle body 2a. In such configuration, the rigidity of the obturator body 3a is uniformly imparted to the needle body 2a. This maintains the needle body 2a in a straight, elongated cylindrical shape while being elastically deformed into a shape that follows the contour of the obturator body 3a. As a result, the needle 2 (i.e., needle body 2a) can be stably punctured into the tissue.

In the embodiments described herein, the groove structure Sg is provided by penetrating a part of the joining area Fc to distribute a sterilizing gas.

In this case, in a state where the needle catheter 1 is exposed to a sterilizing gas atmosphere, the sterilizing gas flows through the joining area Fc where the groove structure Sg is configured. At this time, the sterilizing gas is distributed evenly from between the needle 2 and the obturator 3, which are joined to each other, to also the gap between the obturator body 3a and the needle body 2a. This allows the sterilizing gas to spread to every corner of the needle catheter 1 without leakage. As a result, the entire front and back surfaces of both the needle 2 and the obturator 3 can be fully sterilized.

In conventional types of needle catheters, where there is no groove structure Sg as in the conventional type, it is not possible to assemble the needle 2 and the obturator 3 before sterilization. In the embodiments described herein however, where there is a groove structure Sg, it is possible to assemble the needle 2 and the obturator 3 before sterilization. This allows to dramatically improve the manufacturing efficiency of the needle catheter 1.

In the embodiments described herein, a visually identifiable identification mark (see hatching in FIG. 4) is added to the locking collar 4. This allows the type of needle 2, i.e., the total length of the needle body 2a, to be accurately identified in a short time just by looking at the locking collar 4. As a result, the use efficiency of the needle catheter 1 can be dramatically improved.

In the embodiments described herein, even in a case where the needle 2 (needle body 2a, needle hub 2b) is molded from a material (e.g., polyacetal (POM)) that is extremely difficult to bond to other members, the flair structure Sf that protrudes outward from the needle body 2a is configured at the proximal end portion 2e of the needle body 2a, and the needle hub 2b is configured to cover this flair structure Sf by insert molding a resin material. In such configurations, the flair structure Sf bites into the insert-molded needle hub 2b due to an anchor effect. This maintains the needle body 2a in a state where it is prevented from coming off the needle hub 2b, and as a result, the needle body 2a can be securely coupled to the needle hub 2b.

Additional modifications to the embodiments described herein

In the embodiments described above, a variation in which the groove structure Sg is provided in the obturator hub 3b was described; however, instead of this, for example, a variation in which the groove structure is provided in the needle hub 2b (i.e., the inner circumference of the needle hub 2b in the joining area Fc), or a variation in which the groove structure is provided in both the obturator hub 3b and the needle hub 2b (i.e., both the outer circumference of the obturator hub 3b and the inner circumference of the needle hub 2b in the joining area Fc) is also included in the technical scope of the present invention.

In the embodiments described above, a variation in which a plurality of grooves 8 are arranged as the groove structure Sg was described; however, instead of this, for example, a variation in which one (a single) groove 8 is arranged is also included in the technical scope of the present invention. In this case, the one (single) groove 8 should be arranged along the direction in which the obturator 3 is inserted into the needle 2 (parallel, substantially parallel) in a manner penetrating the joining area Fc.

In the embodiments described above, a variation in which the flair structure Sf protrudes toward a direction between the first direction D1 and the second direction D2 was described as its protruding form; however, instead of this, for example, a variation in which the flair structure Sf protrudes outward along the first direction D1 orthogonal to the needle body 2a, or a variation in which the flair structure Sf protrudes outward toward a direction between the first direction D1 and the third direction D3 from the proximal end portion 2e to the distal end portion 2t is also included in the technical scope of the present invention.

While an embodiments and certain modifications of the present invention have been described, these embodiments and modifications have been presented by way of example only, and are not intended to limit the scope of the inventions. The embodiment and modifications described herein may be embodied in a variety of other forms; furthermore, various omissions, substitutions and changes in the form of the embodiment described herein may be made without departing from the spirit of the invention. The accompanying claims and their equivalents are intended to cover such embodiment or modifications as would fall within the scope and spirit of the inventions.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A needle catheter comprising:
(a) a needle configured to be punctured into tissue, wherein the needle includes:
a hollow and long needle body that is molded of a translucent resin material, having a first proximal end portion and a first distal end portion that is pointed and closed at one end; and
a needle hub surrounding a proximal end portion of the needle body;
wherein the needle body comprises:
an interior lumen that continuously extends from the first proximal end portion of the needle body to a second distal end portion of the needle body, wherein the second distal end portion is within the first distal portion; and
wherein the lumen comprises a distal end having a closed end with a rounded or arc-shape; and
(b) an obturator that is configured to be extractably inserted into the lumen of the needle body.

2. The needle catheter of claim 1, wherein the obturator comprises a distal end in a rounded or arc-shape, whereby when the obturator is inserted into the lumen of the needle body, the surface at the distal end of the obturator contacts the closed end of the lumen of the needle body, thereby providing rigidity to the needle body.

3. The needle catheter of claim 2, wherein the obturator and the closed end of the lumen are arc-shaped and have a substantially similar radius or curvature.

4. The needle catheter of claim 3, wherein the radius of the arc-shape is no less than 0.35 mm.

5. The needle catheter of claim 1, further comprising a locking collar, wherein the locking collar secures the obturator and the needle when the obturator is inserted into the lumen of the needle body.

6. The needle catheter of claim 4, wherein the locking collar (a) is fastened to the needle hub by rotating the locking collar when the obturator is inserted into the needle body lumen and/or (b) is rotatable along the outer circumference of the obturator hub.

7. The needle catheter of claim 1, wherein the obturator comprises an obturator body and an obturator hub at the proximal end of the obturator body, wherein the obturator hub has a larger diameter than the obturator body.

8. The needle catheter of claim 7, wherein the obturator body comprises a straight extended shape, a bent and extended shape, or an arc-like extended shape.

9. The needle catheter of claim 7, wherein the obturator hub and the needle hub are configured to be mutually joined when the obturator body is inserted into the lumen of the needle body.

10. The needle catheter of claim 9, wherein the needle hub is molded from a resin or polyacetal (POM).

11. The needle catheter of claim 5, wherein the locking collar has one or more identification marks on its outer surface to indicate the length of the needle.

12. The needle catheter of claim 1, wherein one or both of the needle and the obturator comprise a groove structure to allow flow of a sterilizing gas.

13. The needle catheter of claim 1, further comprising a flair structure that couples the proximal end portion of the needle body to the needle hub.

14. The needle catheter of claim 13, wherein the flair structure protrudes outward from the proximal end portion of the needle body continuously or intermittently along the outer circumference of the needle body.

15. The needle catheter of claim 1, wherein the needle body is translucent.

16. A system comprising the needle catheter of claim 15, and a diffuser configured to emit light.

17. A method of administering of administering phototherapy or photoimmunotherapy to a tissue, comprising:
inserting the needle catheter of claim 15 by puncturing the tissue;
withdrawing the obturator and retaining the needle in the tissue;
inserting a diffuser into the needle; and irradiating the tissue.

18. The method of claim 17, wherein the tissue comprises a tumor or lesion.

19. The method of claim 17, wherein the method further comprises administering a light-activatable therapy or a light-activatable drug.

20. The method of claim 17, wherein the diffuser is a front-irradiating diffuser or a side-irradiating diffuser and wherein the needle body is translucent.
